(19) United States
(12) Patent Application Publication
(10) Pub. No.: US 2007/0225761 A1
(43) Pub. Date: Sep. 27, 2007

(54) Localized Delivery of A Therapeutic Agent by Barbed Staples

(76) Inventor: Dhanuraj Shetty, Somerset, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/614,955

(22) Filed: Dec. 21, 2006

Related U.S. Application Data
(63) Continuation of application No. 11/388,654, filed on Mar. 24, 2006.

(51) Int. Cl. A61B 17/08

(52) U.S. Cl. 606/219

(57) ABSTRACT

A barbed staple having a therapeutic agent.

### CONTINUING DATA

This patent application claims priority from U.S. patent application Ser. No. 11/388,654, filed Mar. 24, 2006, entitled “Barbed Sutures Having a Therapeutic Agent Thereon” (DEP-5680USNP), the specification of which is incorporated by reference in its entirety.

### BACKGROUND OF THE INVENTION

A barbed staple comprises sharp-edged, resilient protrusions that form acute angles relative to the staple main body. The point of the protrusion faces a direction that is opposite the direction of the staple's path through tissue, so as to anchor the barbs in the tissue when the staple is pulled against the direction of the staple path. Barbed staples enable knotless methods of anchoring staples into tissue and have found applications in plastic and reconstructive surgery (Lee, Arch. Facial Plast. Surg., 7, 55-61, 2005) as well as flexor and Achilles tendon repair (McKenzie, JBJS Br., 49, 440-7, 1967 & Motta, Am. J. Sports Med., 25, 172-6, 1997. Staples are also used for linear lacerations of the torso and extremeties.

Other advantages of stapling includes ease of use, rapidity, cost effectiveness and minimal damage to host defenses. Because of the advantages over conventional suturing techniques, mechanical stapling is now widely used for various surgical procedures.

Barbed staples can be made into bi-directional and continuous array designs. In continuous array designs, the barbs point in only one direction along the staple length and are used in wound closure procedures in a manner similar to conventional staples, but without the need of knotting. Bi-directional staple designs include two sets of barbs, one set on either side of the midpoint, wherein the barb sets point at each other and towards the midpoint of the staple. The midpoint of the staple, where the barbs change direction, corresponds to the contact point between the two portions of tissue that are being repaired by the staple.

Numerous types of surgical stapes have been reported. For example, U.S. Pat. No. 3,625,022 (Engel) discloses a device comprised of an outer tube, an inner tube and a locking means. The suture guard is semirigid polyethylene.

U.S. Pat. No. 4,950,285 (Wilk I) and U.S. Pat. No. 5,123,913 (Wilk II) disclose a one-piece suture.

U.S. Pat. No. 5,601,604 (Vincent) discloses a one-piece gastric band comprised of a body portion with a tail end and a buckle. The tail end of the body portion is inserted into the buckle to form a loop.

U.S. Pat. No. 4,534,352 (Korthoff) discloses a two-piece surgical fastener made from an absorbable resinous material. The surgical fastener is comprised of a base and a prong-containing member. Each prong is inserted into an aperture in the base to form a secure connection.

U.S. Pat. No. 4,612,923 (Kronenthal) discloses a two-piece surgical fastener made from a synthetic absorbable polymer containing an absorbable glass filler. The surgical fastener is comprised of a staple and a receiver. The staple is inserted into the openings in the receiver to form a secure connection.

U.S. Pat. No. 4,646,741 (Smith) discloses a two-piece surgical staple made from a blend of a lactide/glycolide copolymer and poly(p-dioxanone). The surgical staple is comprised of a base with two legs and a receiver. The legs of the base are inserted into receptacles in the receiver to form a secure connection.

U.S. Pat. No. 4,889,119 (Jamiolkowski) discloses a two-piece surgical staple made from a glycolide-rich blend of two or more polymers. The surgical staple is comprised of a base with two legs and a receiver. The legs of the base are inserted into receptacles in the receiver to form a secure connection.

U.S. Pat. No. 5,282,829 (Hermes) discloses a two-piece biodegradable surgical device comprised of a fastener with two prongs and a receiver. The prongs of the fastener are inserted into the receiver to form a secure connection. Both the fastener and the receiver contain a hollow core region.

U.S. Pat. No. 5,439,479 (Shichman) discloses a biodegradable two-piece surgical clip comprised of a fastener and a retainer. The fastener has a set of legs containing gripping means adapted to be engaged by the retainer. When the legs of the fastener are engaged by the retainer, a closed connection is formed.

U.S. Pat. No. 5,462,542 (Alesi) discloses a biodegradable one-piece surgical strap assembly having a flexible elongated strap and a buckle attached to one end of the strap. A portion of the strap contains a plurality of ratchet teeth. The ratchet teeth of the strap engage a locking mechanism in the buckle to form a loop.

U.S. Pat. No. 5,549,619 (Peters) discloses a biodegradable one-piece or two-piece surgical device comprising an eye with a latching pawl and a flexible strip with ratchet teeth. The ratchet teeth of the flexible strip engage with the latching pawl of the eye to form a loop.

U.S. Pat. No. 5,643,295 (Yoon) discloses an apparatus for suturing tissue comprising a knotting element connected between two length portions of filamentous suture material to form a contractile loop for confining segments of the length portions therein.

U.S. Pat. No. 4,204,623 (Green) discloses a manually powered surgical stapling instrument for applying sterilized staples to disunited skin or fascia. A pusher is slidably mounted in the cartridge for advancing the staples, for ejecting the staples, and for forming the staples around an anvil.

U.S. Pat. No. 4,489,875 (Crawford) discloses an instrument for applying staples to skin by bending the staple around an anvil. As the staple is forced against the anvil, the staple bends and the legs penetrate the tissue and apply closing pressure across the wound.

Recent advances in barbed suture technology have been reviewed by Leung, in “Advances in Biomedical Textiles and Healthcare Products”, 62-90, 2004. This review article outlines surgical techniques , holding strengths, and in vivo performance of such sutures. The design of conventional barbed sutures is also reported in U.S. Pat. No. 3,123,077 (“Alcamo”); U.S. Pat. No. 5,053,047 (Yoon”); and U.S. Pat. No. 5,342,376 (“Ruff”). Surgical methods using barbed sutures are reported in U.S. Pat. No. 5,931,855 (“Buncke”). None of these references disclose a barbed suture having a therapeutic agent coated thereon.

Conventional sutures coated with therapeutic agents have been reported in the literature sutures coated with collagen, butyric acid and a variety of growth factors have been used in soft tissue repair. Mazzocca, AAOS 2005, #338; Wright, 50^{th} ORS, 2004, poster #1234; Petersen, 51^{st} ORS 2005, paper number 0076; Schmidmaier, J. Biomed. Mat. Res. (Appl. Biomat.) 58, 449-55, 2001; and Rickert, Growth Factors, 19, 115-26, 2001. These studies have shown promising in vitro and in vivo data.

Sutures coated with antibiotics are clinically available. At present, VICRYL Plus Coated Suture (Ethicon, Somerville, N.J.) is the first and only antibacterial suture cleared by the FDA for inhibiting the colonization of bacteria that cause the majority of surgical site infections (Rothburger, Surgical Infection Society Journal (Suppl) December 2002). VICRYL Plus Coated Sutures contain IRGACARE MP*, the most pure form of triclosan, a proven broad-spectrum antibacterial used effectively in consumer products for more than 30 years. VICRYL Plus Coated Suture is indicated for use in general soft tissue approximation and/or ligation, except ophthalmic, cardiovascular and neurological tissues.

### SUMMARY OF THE INVENTION

The present invention relates to a barbed staple comprising a therapeutic agent. Self-anchoring staples, such as those with bi-directional barb designs, facilitate wound closure by anchoring themselves in tissue without the need of additional securement (i.e., knots). This enables the staple to be passed directly through the wound site. Barbed staples combined with a therapeutic agent (such as recombinant human growth and differentiation factor-5 (rhGDF-5)) would provide a localized delivery of the agent to the wound and an improved healing response. This would be an advantage over conventional staples coated with bioactive therapeutic agents that are typically stitched along the wound edge, thereby requiring the released agent to diffuse through tissue in order to reach the defect site. As the staple is embedded within the tissue, the therapeutic will be concentrated at the defect site and there will also be less loss of the bioactive therapeutic agent to neighboring tissue. The barbed staple also possesses an increased surface area, which allows for a greater amount of therapeutic agent to be absorbed on the surface per given length of staple as compared to conventional staples.

Barbed staples coated with a bioactive therapeutic agent would also have improved wound holding strength, as the therapeutic agent would enhances the quality of the tissue in which the staples are anchored.

Therefore, in accordance with the present invention, there is provided a method of stapling, comprising the steps of:
a) providing a wound defect comprising a first and second tissue planes and a crevice therebetween,
b) providing a barbed staple having a first end portion, a second end portion and an intermediate portion comprising a therapeutic agent, and
c) inserting the first end of the staple into the first tissue plane and the second end portion of the staple into the second tissue plane so that the intermediate portion of the staple having the therapeutic agent thereon contacts the crevice

Also in accordance with the present invention, there is provided a barbed staple comprising a therapeutic agent.

### DESCRIPTION OF THE FIGURES

FIG. 1 shows a barbed staple having an intermediate portion coated with the therapeutic film directed across a wound site.

FIG. 2 shows a barbed staple having a therapeutic sheet at its intermediate portion directed across a wound site, wherein the sheet is aligned parallel to the crevice of the wound.

FIGS. 3a and 3b show a barbed staple having localized depots of therapeutic agent on its outer surface.

### DETAILED DESCRIPTION OF THE INVENTION

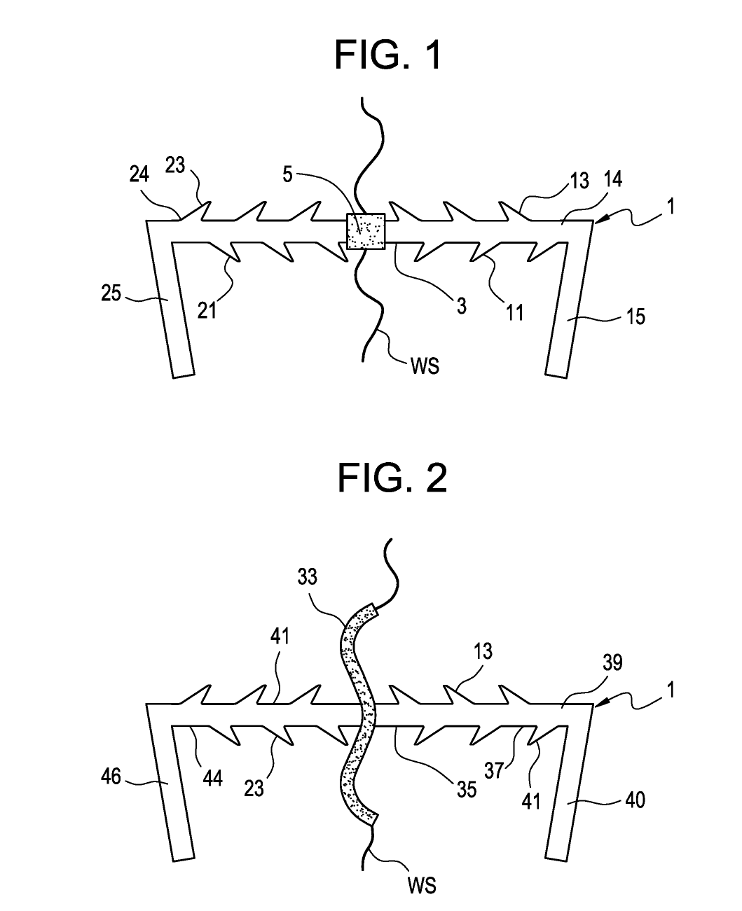

Now referring to FIG. 1, there is provided a barbed staple 1 having an intermediate portion 3, wherein at least part of the intermediate portion is coated with the therapeutic overlay 5 and is directed across a wound site WS. The staple comprises a first barbed portion 11 comprising a first plurality of barbs 13 facing a first direction and having a first end 14, a first leg 15 extending substantially normally from the first end, a second barbed portion 21 comprising a second plurality of barbs 23 facing a second direction and having a second end 24, and a second leg 25 extending substantially normally from the second end and in substantially the same direction as the first leg. In this FIG. 1, the therapeutic overlay contacts only the intermediate portion of the staple.

The therapeutic agent is present in the therapeutic overlay that coats the intermediate portion of the bi-directional staple. This location corresponds to the contact point between the two planes of tissue that are being repaired by the staple.

In another embodiment, the therapeutic coating coats the entire length of the staple. In a preferred embodiment thereof, the concentration of the therapeutic agent is greater in the intermediate portion of the staple than in the first or second barbed portions, and the first or second legs.

Now referring to FIG. 2, there is provided a barbed staple 31 having a therapeutic sheet 33 at its intermediate portion 35 directed across a wound site (WS), wherein the sheet is aligned parallel to the crevice of the wound site. The therapeutic agent contacting the intermediate section is provided within a sheet contacting the intermediate section. The staple comprises a first barbed portion 37 comprising a first plurality of barbs 13 facing a first direction and having a first end 39, a first leg 40 extending substantially normally from the first end, a second barbed portion 41 comprising a second plurality of barbs 23 facing a second direction and having a second end 44, a second leg 46 extending substantially normally from the second end and in substantially the same direction as the first leg. The first 37 and second 41 barbed portions of the staple define a longitudinal axis, and the sheet is disposed in an orientation substantially normal to the longitudinal axis.

The sheet 33 containing a bioactive therapeutic agent is placed within the intermediate section of the bi-directional barbed staple, or can be rolled over the staple surface. The sheet lays perpendicular to the staple and co-exists within the 2-dimensional plane of the wound site. The sheet is preferably attached to the staple by piercing it with a needled end of the staple and then sliding it to the intermediate portion of the staple. The bi-directional staple design will maintain the sheet at the intermediate portion. Preferably, the sheet comprises a material that loses its rigidity when wetted so that it has the ability to mold and conform to the wound site.

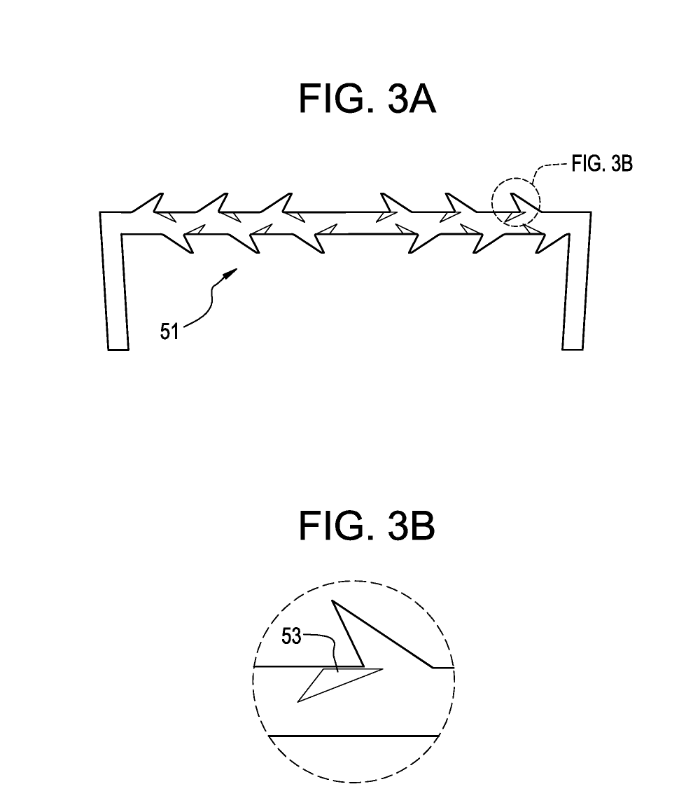

Now referring to FIGS. 3a and 3b, there is provided a barbed staple 51 having localized depots 53 of therapeutic agent on its outer surface. The manufacture of barbed staples can be carried out by the methods disclosed in U.S. Pat. No. 3,123,077 (“Alcamo”); U.S. Pat. No. 5,053,047 (Yoon”); and U.S. Pat. No. 5,342,376 (“Ruff”), the specifications of which are incorporated by reference in their entireties. Barbed staples are typically produced by micro-machining a monofilament staple leaving defects along the staple core. These defects can be used as depots for therapeutic agents. Other methods include the use of a laser and fraying. The depots can be filled by a microfilling process or a dipcoating followed by a wipe of the staple core. The barbed staples of these embodiments can be either a continuous array type or a bi-directional type.

In another embodiment, (not shown), there is provided a blend of resorbable synthetic polymer and therapeutic agent that has been molded into a staple. This staple is then subsequently micro-machined to yield the barbed staple, having either a continuous array design or a bi-directional design. The therapeutic agent is released as the staple material degrades in the physiological environment.

In some preferred embodiments, the therapeutic agent to be coated upon the staple is a protein. In some embodiments, the therapeutic protein to be coated upon the staple is selected from the group consisting of growth factors, anti-microbials, analgesics, anti-inflammatory agents, anti-neoplastics, RGD sequences, fibrin and clotting factors.

In some embodiments, the therapeutic agent to be coated upon the staple is selected from the group consisting of amino acids, anabolics, analgesics and antagonists, anaesthetics, anti-adrenergic agents, anti-asthmatics, anti-atherosclerotics, antibacterials, anticholesterolics, anti-coagulants, antidepressants, antidotes, anti-emetics, anti-epileptic drugs, anti-fibrinolytics, anti-inflammatory agents, antihypertensives, antimetabolites, antimigraine agents, antimycotics, antinauseants, antineoplastics, anti-obesity agents, antiprotozoals, antipsychotics, antirheumatics, antiseptics, antivertigo agents, antivirals, appetite stimulants, bacterial vaccines, bioflavonoids, calcium channel blockers, capillary stabilizing agents, coagulants, corticosteroids, detoxifying agents for cytostatic treatment, diagnostic agents (like contrast media, radiopaque agents and radioisotopes), electrolytes, enzymes, enzyme inhibitors, ferments, ferment inhibitors, gangliosides and ganglioside derivatives, hemostatics, hormones, hormone antagonists, hypnotics, immunomodulators, immunostimulants, immunosuppressants, minerals, muscle relaxants, neuromodulators, neurotransmitters and neurotrophins, osmotic diuretics, parasympatholytics, para-sympathomimetics, peptides, proteins, psychostimulants, respiratory stimulants, sedatives, serum lipid reducing agents, smooth muscle relaxants, sympatholytics, sympathomimetics, vasodilators, vasoprotectives, vectors for gene therapy, viral vaccines, viruses, vitamins, oligonucleotides and derivatives, saccharides, polysaccharides, glycoproteins, hyaluronic acid, and any excipient that can be used to stabilize a proteinaceous therapeutic

In some embodiments, the therapeutic agent to be coated upon the staple is a non-curing therapeutic agent.

As used herein, the term “growth factors” encompasses any cellular product that modulates the adhesion, migration, proliferation, or differentiation of other cells, particularly connective tissue progenitor cells. The growth factors that may be used in accordance with the present invention include, but are not limited to, members of the fibroblast growth factor family, including acidic and basic fibroblast growth factor (FGF-1 and -2) and FGF-4, members of the platelet-derived growth factor (PDGF) family, including PDGF-AB, PDGF-BB and PDGF-AA; Epidermal Growth Factors (EGFs), members of the insulin-like growth factor (IGF) family, including IGF-I and -II; the Transforming Growth Factor (TGF-β) superfamily, including TGF-β1, 2 and 3 (including rhGDF-5), osteoid-inducing factor (OIF), angiogenin(s), endothelins, hepatocyte growth factor and keratinocyte growth factor; members of the bone morphogenetic proteins (BMP's) BMP-1, (BMP-3); BMP-2; OP-1; BMP-2A, -2B, and -7, BMP-14; Heparin Binding Growth Factors HBGF-1 and -2; growth differentiation factors (GDF's), members of the hedgehog family of proteins, including indian, sonic and desert hedgehog; ADMP-1; members of the interleukin (IL) family, including IL-1 thru -6; members of the colony-stimulating factor (CSF) family, including CSF-1, G-CSF, GM-CSF, VEGF integrin binding sequence, ligands, bone morphogenic proteins, epidermal growth factor, IGF-I, IGF-II, TGF-β I-III, growth differentiation factor, parathyroid hormone, hyaluronic acid, glycoprotein, lipoprotein, small molecules that affect the upregulation of specific growth factors, tenascin-C, fibronectin, thromboelastin, thrombin-derived peptides, heparin-binding domains, and isoforms thereof.

In some embodiments, the growth factor is GDF-5, preferably rhGDF-5. More preferably, the rhGDF-5 is administered using a solution with concentrations between 10 ng/mL and 40 mg/mL, more preferably between 100 ng/mL and 10 mg/mL, most preferably between 1 μg/mL and 5 mg/mL.

Any biocompatible fluid capable of coating a staple may be used in accordance with the present invention. Suitable fluids include aqueous liquids (such as saline) and gels that include, but are not limited to, hyaluronic acid, succinalyted collagen, carboxymethyl cellulose (CMC), gelatin, collagen gel, fibrinogen/thrombin, solvents such as ethanol, any excipient that can be used to stabilize a proteinaceous therapeutic and liquid polymers (MGSA).

Preferably, the staples used in accordance with the present invention will be bioresorbable. However, the staples may also be non-resorbable. Preferred bioresorbable materials, which can be used to make the staples of the present invention, include bioresorbable polymers or copolymers, preferably selected from the group consisting of hydroxy acids, (particularly lactic acids and glycolic acids; caprolactone; hydroxybutyrate; dioxanone; orthoesters; orthocarbonates; and aminocarbonates). Preferred bioresorbable materials also include natural materials such as chitosan, collagen, cellulose, fibrin, hyaluronic acid; fibronectin, and mixtures thereof. However, synthetic bioresorbable materials are preferred because they can be manufactured under process specifications which insure repeatable properties.

Synthetic nonresorbable materials include silk, cotton, linen, nylon, polypropylene, polybutester, nylon and polyester.

A variety of bioabsorbable polymers can be used to make the staple of the present invention. Examples of suitable biocompatible, bioabsorbable polymers include but are not limited to polymers selected from the group consisting of aliphatic polyesters, poly(amino acids), copoly(ether-esters), polyalkylenes oxalates, polyamides, tyrosine derived polycarbonates, poly(iminocarbonates), polyorthoesters, polyoxaesters, polyamidoesters, polyoxaesters containing amine groups, poly(anhydrides), polyphosphazenes, biomolecules (i.e., biopolymers such as collagen, elastin, bioabsorbable starches, etc.), polyurethanes, and blends thereof. For the purpose of this invention aliphatic polyesters include, but are not limited to, homopolymers and copolymers of lactide (which includes lactic acid, D-,L- and meso lactide), glycolide (including glycolic acid), ε-caprolactone, p-dioxanone (1,4-dioxan-2-one), trimethylene carbonate (1,3-dioxan-2-one), alkyl derivatives of trimethylene carbonate, δ-valerolactone, β-butyrolactone, χ-butyrolactone, ε-decalactone, hydroxybutyrate, hydroxyvalerate, 1,4-dioxepan-2-one (including its dimer 1,5,8,12-tetraoxacyclotetradecane-7,14-dione), 1,5-dioxepan-2-one, 6,6-dimethyl-1,4-dioxan-2-one, 2,5-diketomorpholine, pivalolactone, χ,χ-diethylpropiolactone, ethylene carbonate, ethylene oxalate, 3-methyl-1,4-dioxane-2,5-dione, 3,3-diethyl-1,4-dioxan-2,5-dione, 6,8-dioxabicycloctane-7-one and polymer blends thereof. Poly(iminocarbonates), for the purpose of this invention, are understood to include those polymers as described by Kemnitzer and Kohn, in the Handbook of Biodegradable Polymers, edited by Domb, et. al., Hardwood Academic Press, pp. 251-272 (1997). Copoly(ether-esters), for the purpose of this invention, are understood to include those copolyester-ethers as described in the Journal of Biomaterials Research, Vol. 22, pages 993-1009, 1988 by Cohn and Younes, and in Polymer Preprints (ACS Division of Polymer Chemistry), Vol. 30(1), page 498, 1989 by Cohn (e.g. PEO/PLA). Polyalkylene oxalates, for the purpose of this invention, include those described in U.S. Pat. Nos. 4,208,511; 4,141,087; 4,130,639; 4,140,678; 4,105,034; and 4,205,399. Polyphosphazenes, co-, ter- and higher order mixed monomer-based polymers made from L-lactide, D,L-lactide, lactic acid, glycolide, glycolic acid, para-dioxanone, trimethylene carbonate and ε-caprolactone such as are described by Allcock in The Encyclopedia of Polymer Science, Vol. 13, pages 31-41, Wiley Intersciences, John Wiley & Sons, 1988 and by Vandorpe, et al in the Handbook of Biodegradable Polymers, edited by Domb, et al, Hardwood Academic Press, pp. 161-182 (1997). Polyanhydrides include those derived from diacids of the form HOOC—C₆H₄—O—(CH₂)m-O—C₆H₄—COOH, where m is an integer in the range of from 2 to 8, and copolymers thereof with aliphatic alpha-omega diacids of up to 12 carbons. Polyoxaesters, polyoxaamides and polyoxaesters containing amines and/or amido groups are described in one or more of the following U.S. Pat. Nos. 5,464,929; 5,595,751; 5,597,579; 5,607,687; 5,618,552; 5,620,698; 5,645,850; 5,648,088; 5,698,213; 5,700,583; and 5,859,150. Polyorthoesters such as those described by Heller in Handbook of Biodegradable Polymers, edited by Domb, et al, Hardwood Academic Press, pp. 99-118 (1997).

Preferably, the bioresorbable material is selected from the group consisting of poly(lactic acid) (“PLA”) and poly(glycolic acid)(“PGA”), and copolymers thereof.

In some of the preferred embodiments, one of the resorbable staples is selected from the group consisting of PLA, PGA, polydioxanone (PDO), polycaprolactone (PCL), and mixtures thereof.

In some embodiments, the staples may comprise shape memory materials such as shape memory polymers and shape memory metals, such as nitinol.

In some preferred embodiments, the staple comprises collagen because rhGDF-5 has a high affinity towards collagen. In some preferred embodiments, the staple comprises surgical gut, which comprises purified connective tissue (of which its main component is type I collagen) derived from either the serosal layer or the submucosal fibrous layer of bovine intestines.

In some embodiments, there is provided a resorbable composite comprising a first resorbable barbed staple and a second resorbable barbed staple, wherein the first resorbable staple is made of a material different than the second resorbable staple, and wherein at least one of the staples is coated with a therapeutic agent, preferably a growth factor. Preferably, each staple is coated with the growth factor. Preferably, the growth factor is a BMP. More preferably, the growth factor is rhGDF-5. In some embodiments, one of the resorbable staples is PLGA.

In other embodiments, there is provided a partially resorbable composite comprising a first resorbable barbed staple and a second non-resorbable barbed staple, wherein at least one of the staples is coated with a growth factor. Preferably, each staple is coated with the growth factor, wherein the growth factor is preferably a BMP. More preferably, the growth factor is rhGDF-5. In some preferred embodiments thereof, the resorbable staple is polydioxanone and the non-resorbable staple is polyethylene. More preferably, the growth factor is coated upon the composite staple disclosed in US Published Patent Application No. US 2005/0149118 (Koyfman), the specification is incorporated by reference in its entirety. In some embodiments, this composite is ORTHOCORD, available from Mitek, Raynham, Mass.

In other embodiments, there is provided a non-resorbable composite comprising a first non-resorbable barbed staple and a second non-resorbable barbed staple, wherein the first non-resorbable staple is made of a material different than the second non-resorbable staple, and wherein at least one of the staples is coated with a therapeutic agent, preferably a growth factor. Preferably, each staple is coated with the growth factor. Preferably, the growth factor is a BMP. More preferably, the growth factor is rhGDF-5. In some preferred embodiments thereof, the first non-resorbable staple is a polyethylene core, and the second non-resorbable staple is a polyester braided jacket. More preferably, the growth factor is coated upon the composite staple disclosed in U.S. Pat. No. 6,716,234 (“Grafton”), the specification is incorporated by reference in its entirety. In some embodiments, this composite is FIBERWIRE, available from Arthrex, Naples, Fla.

In other embodiments, there is provided a composite staple comprising of a resorbable barbed staple and a resorbable conventional staple(s), wherein the barbed staple is made of a material different then the conventional staple, wherein the conventional staple(s) is braided around the barbed staple, and wherein at least one of the staples is coated with a therapeutic agent, preferably a growth factor. Preferably, each staple is coated with the growth factor. Preferably, the growth factor is a BMP. More preferably, the growth factor is rhGDF-5. In some embodiments, one of the resorbable staples is PLGA.

In other embodiments, there is provided a composite staple comprising of a non-resorbable barbed staple and a non-resorbable conventional staple(s), wherein the barbed staple is made of a material different then the conventional staple, wherein the conventional staple(s) is braided around the barbed staple, and wherein at least one of the staples is coated with a therapeutic agent, preferably a growth factor. Preferably, each staple is coated with the growth factor. Preferably, the growth factor is a BMP. More preferably, the growth factor is rhGDF-5.

In other embodiments, there is provided a composite staple comprising of a non-resorbable barbed staple and a resorbable conventional staple(s), wherein the conventional staple(s) is braided around the barbed staple, and wherein at least one of the staples is coated with a therapeutic agent, preferably a growth factor. Preferably, each staple is coated with the growth factor. Preferably, the growth factor is a BMP. More preferably, the growth factor is rhGDF-5. In some embodiments, the resorbable conventional staples are PLGA.

In some embodiments of the present invention, the wound defect that is treated by the barbed staple of the present invention is selected from the group consisting of an anterior cruciate ligament defect, a medial collateral ligament defect, a meniscal defect, a rotator cuff defect, a defect in an annulus fibrosus of an intervertebral disc, a dna ligament. The preferred therapeutic agent therefore is a growth factor, more preferably GDF-5.

We claim:

1. A barbed staple comprising a therapeutic agent.

2. The staple of claim 1 having an outer surface, wherein the therapeutic agent contacts at least a portion of the outer surface of the staple.

3. The staple of claim 1 having a first barbed portion, a second barbed portion and an intermediate portion, wherein the therapeutic agent contacts the intermediate portion of the staple.

4. The staple of claim 3 wherein the therapeutic agent contacts only the intermediate portion of the staple.

5. The staple of claim 3 wherein the first barbed portion comprises a first plurality of barbs facing a first direction, and the second barbed portion comprises a second plurality of barbs facing a second direction.

6. The staple of claim 3 wherein the first barbed portion comprises a first plurality of barbs facing a first direction, and the second barbed portion comprises a second plurality of barbs facing the first direction.

7. The staple of claim 3 wherein the therapeutic agent contacting the intermediate section is provided within a sheet contacting the intermediate section.

8. The staple of claim 7 wherein the first and second barbed portions of the staple define a longitudinal axis, and the sheet is disposed in an orientation substantially normal to the longitudinal axis.

9. The staple of claim 8 wherein the sheet comprises a material that loses rigidity when wetted.

10. The staple of claim 1 wherein the therapeutic agent is a growth factor.

11. The staple of claim 10 wherein the growth factor is a member of the BMP superfamily.

12. The staple of claim 10 wherein the growth factor is a growth and differentiation factor (GDF).

13. The staple of claim 1 wherein the therapeutic agent coats an entire length of the staple.

14. The staple of claim 13 having a first barbed portion, a second barbed portion and an intermediate portion, wherein the therapeutic agent coats the intermediate portion of the staple at a first concentration and wherein the therapeutic agent coats the first and second barbed portions at a second concentration, wherein the first concentration in the intermediate portions is greater than the second concentration in the end portions.

15. The staple of claim 1 wherein the therapeutic agent is provided in localized depots upon an outer surface of the staple.

16. The staple of claim 15 wherein the depots comprised machined defects in the staple.

17. A method of suturing, comprising the steps of:
a) providing a wound defect comprising a first and second tissue planes and a crevice therebetween,
b) providing a barbed staple having a first barbed portion having a first leg extending therefrom, a second barbed portion having a second leg extending therefrom and an intermediate portion comprising a therapeutic agent, and
c) inserting the first leg of the staple into the first tissue plane and the second leg of the staple into the second tissue plane so that the intermediate portion of the staple having the therapeutic agent thereon contacts the crevice.

18. The method of claim 17 wherein the therapeutic agent contacts only the intermediate portion of the staple.

19. The method of claim 17 wherein the first barbed portion comprises a first plurality of barbs facing a first direction, and the second barbed portion comprises a second plurality of barbs facing a second direction.

20. The method of claim 19 wherein the therapeutic agent contacts only the intermediate portion of the staple.

21. The method of claim 19 wherein the therapeutic agent contacting the intermediate section is provided within a sheet contacting the intermediate section.

22. The method of claim 21 wherein the sheet is inserted into the crevice.

23. The method of claim 17 wherein the therapeutic agent is a growth factor.

24. The method of claim 23 wherein the growth factor is a member of the BMP superfamily.

25. The method of claim 23 wherein the growth factor is a growth and differentiation factor (GDF).

26. The method of claim 17 wherein the defect is an anterior cruciate ligament defect.

27. The method of claim 26 wherein the therapeutic agent is a growth factor.

28. The method of claim 27 wherein the growth factor is GDF-5.

29. The method of claim 17 wherein the defect is a medial collateral ligament defect.

30. The method of claim 29 wherein the therapeutic agent is a growth factor.

31. The method of claim 30 wherein the growth factor is GDF-5.

32. The method of claim 17 wherein the defect is a meniscal defect.

33. The method of claim 32 wherein the therapeutic agent is a growth factor.

34. The method of claim 33 wherein the growth factor is GDF-5.

35. The method of claim 17 wherein the defect is a rotator cuff defect.

36. The method of claim 35 wherein the therapeutic agent is a growth factor.

37. The method of claim 36 wherein the growth factor is GDF-5.

38. The method of claim 17 wherein the soft tissue is an annulus fibrosus of an intervertebral disc.

39. The method of claim 38 wherein the therapeutic agent is a growth factor.

40. The method of claim 39 wherein the growth factor is GDF-5.

41. The method of claim 17 wherein the soft tissue is a ligament.

42. The method of claim 41 wherein the therapeutic agent is a growth factor.

43. The method of claim 42 wherein the growth factor is GDF-5.

44. A device comprising:
i) a staple comprising an intermediate section and first and second barbed portions, and
ii) a sheet comprising a therapeutic agent, wherein the sheet contacts the intermediate section of the staple.

45. The device of claim 44 wherein the first and second barbed portions of the staple define a longitudinal axis, and the sheet is disposed in an orientation substantially normal to the longitudinal axis.

46. The device of claim 44 wherein the sheet comprises a material that loses rigidity when wetted.
